(19) United States
(12) Patent Application Publication
(10) Pub. No.: US 2017/0105867 A1
(43) Pub. Date: Apr. 20, 2017

(54) Remotely Operated Snore-Stopping Pillow

(71) Applicant: Hung-Nan Hsieh, Taoyuan City (TW)

(72) Inventor: Hung-Nan Hsieh, Taoyuan City (TW)

(21) Appl. No.: 15/200,067

(22) Filed: Jul. 1, 2016

(30) Foreign Application Priority Data
Oct. 16, 2015 (TW) 104216548

(51) Int. Cl. A61F 5/56; G06F 3/16; G08C 17/02; A47G 9/10

(52) U.S. Cl. CPC A61F 5/56; A47G 9/1027; G06F 3/16; G08C 17/02; G08C 2201/93

(57) ABSTRACT

A remotely operated snore-stopping pillow includes a pillow, a wired controller, and a wireless controller. The pillow includes a body receiving a bladder. The wired controller includes a wireless receiver, a high/low controller for the pillow, and a control valve. The wireless receiver is connected to the high/low controller. The control valve is connected to an air pump and a sound sensor. The sound sensor is connected to a snore-stopping function controller. The high/low controller is connected to the control valve. The control valve is connected to the bladder of the pillow via an air tube. The wireless controller includes a wireless transmitter, a high/low adjustment signal generator connected to the wireless transmitter, and a snore-stopping function signal generator.

### BACKGROUND OF THE INVENTION

The present invention relates to a remotely operated snore-stopping pillow and, more particularly, to a pillow that can be wirelessly controlled to control the height of the pillow and to activate a snore-stopping function.

Snore is a common phenomenon during sleep. Applicant of the present invention has invented a snore-preventing memory pillow with cervical vertebra adjustment and has filed a patent application which has been registered as Taiwan Utility Model No. M461386.

However, the above snore-preventing memory pillow with cervical vertebra adjustment still has disadvantages. Firstly, the snore-stopping function can only be operated through a control box, such that an operator has to come close the pillow to operate the control box. If the user of the pillow forgets to activate the snore-stopping function, another person must come to the user to activate the snore-stopping function, which is inconvenient. Secondly, the sensitivity of a sound collector of the control box cannot be adjusted, but the volumes of snores of different users are different. If the sound collector has a high sensitivity, the snore-stopping function is often activated by the surround sound.

Thus, a need exists for a novel device that mitigates and/or obviates the above disadvantages.

### BRIEF SUMMARY OF THE INVENTION

The primary objective of the present invention is to provide a wirelessly controlled pillow permitting ease of use by an operator. Furthermore, it is not necessary to place the wired controller near the user of the pillow, reducing the affect by electromagnetic waves and avoiding interruption of the sleep of the user of the pillow. Thus, the pillow can be used in medical therapy.

Another objective of the present invention is to adjust the sensitivity of a sound sensor to suit different users.

To fulfill the above objectives, the present invention provides a remotely operated snore-stopping pillow including a pillow, a wired controller, and a wireless controller. The pillow includes a body receiving a bladder. The wired controller includes a wireless receiver, a high/low controller for the pillow, and a control valve. The wireless receiver is connected to the high/low controller. The control valve is connected to an air pump and a sound sensor. The sound sensor is connected to a snore-stopping function controller. The high/low controller is connected to the control valve. The control valve is connected to the bladder of the pillow via an air tube. The wireless controller includes a wireless transmitter, a high/low adjustment signal generator connected to the wireless transmitter, and a snore-stopping function signal generator.

The wireless receiver of the wired controller and the wireless transmitter of the wireless controller can transmit control signals via a blue-tooth wireless transmission interface or a wireless local area network interface.

The wireless controller can be a smart mobile communication device, and the high/low adjustment signal generator and the snore-stopping function signal generator can be generated by an application software.

The sound sensor of the wired controller can be connected to a sensitivity adjuster. The wireless transmitter can be connected to a sensitivity adjustment signal generator. The sensitivity adjustment signal generator can be generated by the application software.

The present invention will become clearer in light of the following detailed description of illustrative embodiments of this invention described in connection with the drawings.

### DESCRIPTION OF THE DRAWINGS

FIG. 1 is a block diagram of a remotely operated snore-stopping pillow according to the present invention.

FIG. 2 is a diagrammatic perspective view of a pillow and a wired controller of the remotely operated snore-stopping pillow according to the present invention.

FIG. 3 is a diagrammatic view of a wireless controller of the remotely operated snore-stopping pillow according to the present invention.

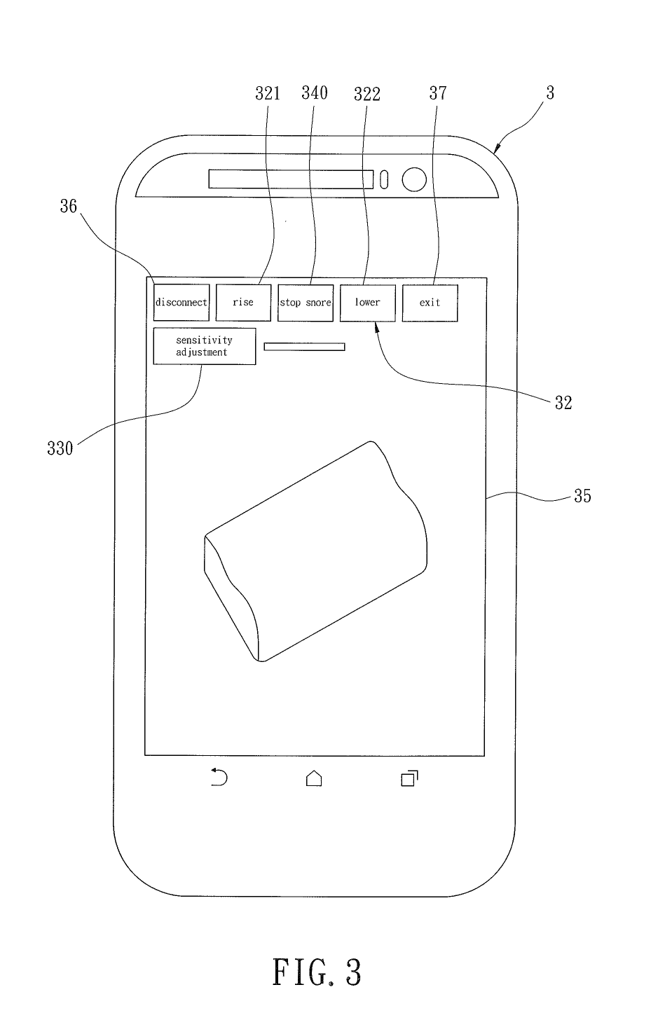

### DETAILED DESCRIPTION OF THE INVENTION

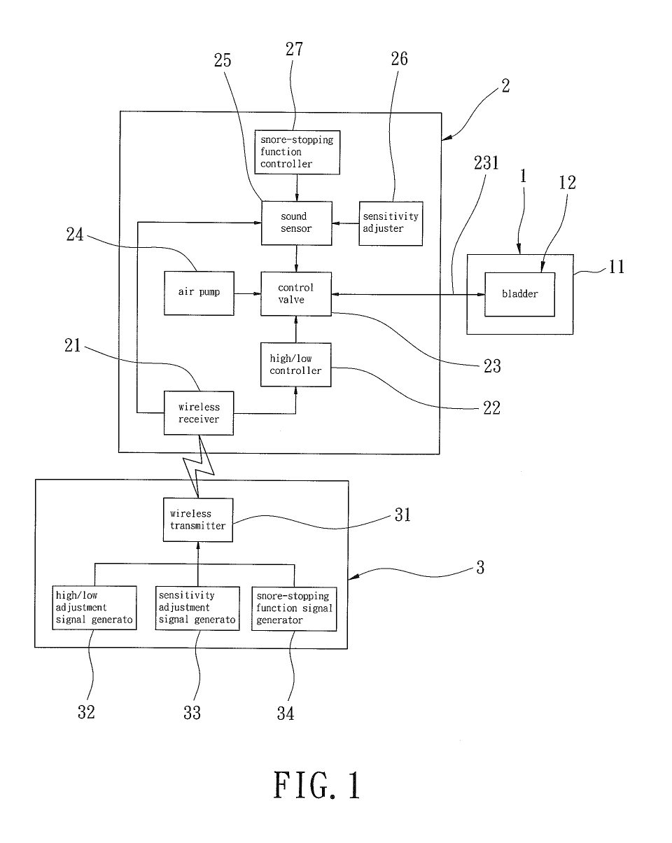

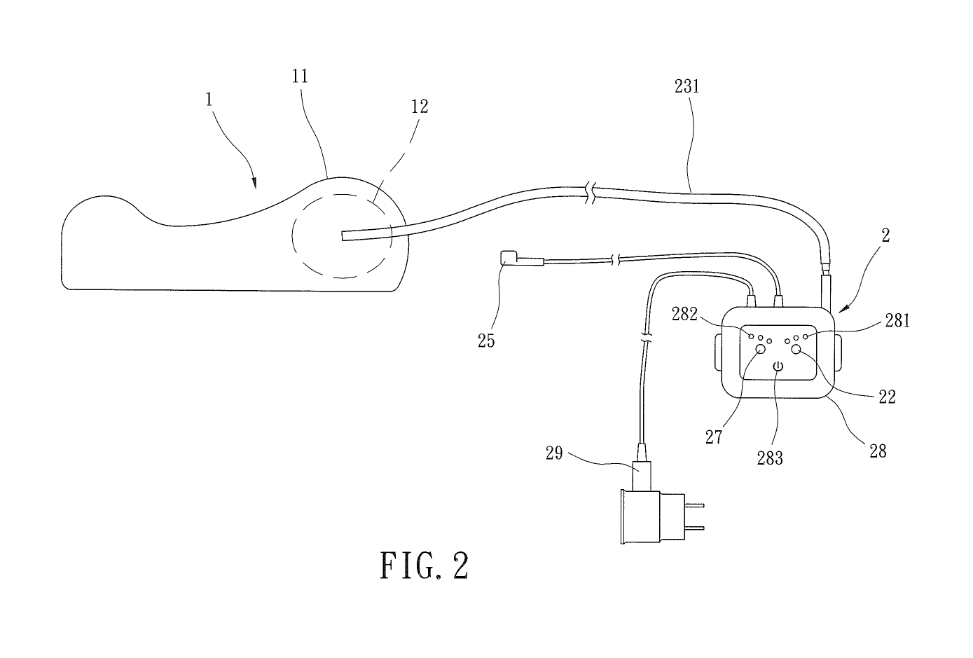

With reference to FIGS. 1 and 2, a remotely operated snore-stopping pillow according to the present invention includes a pillow 1, a wired controller 2, and a wireless controller 3.

The pillow 1 includes a body 11 receiving a bladder 12.

The wired controller 2 includes a wireless receiver 21, a high/low controller 22 for the pillow 1, and a control valve 23. The wireless receiver 21 is connected to the high/low controller 22. The control valve 23 is connected to an air pump 24 and a sound sensor 25. The sound sensor 25 is connected to a sensitivity adjuster 26 and a snore-stopping function controller 27. The high/low controller 22 is connected to the control valve 23. The control valve 23 is connected to the bladder 12 of the pillow 1 via an air tube 231. The wireless receiver 21, the high/low controller 22, the control valve 23, the air pump 24, the sensitivity adjuster 26, and the snore-stopping function controller 27 are mounted in a control box 28. The control box 28 includes a high/low position indicating lamp 281 for the pillow 1, a snore-stopping position indicating lamp 282, and a power switch 283. The control box 28 is connected by an electric wire 29 to an external power source for providing electricity to the wired controller 2. Furthermore, the sound sensor 25 is disposed outside of the controller 28 and is located adjacent to the pillow 1.

The wireless controller 3 includes a wireless transmitter 31, a high/low adjustment signal generator 32 connected to the wireless transmitter 31, a sensitivity adjustment signal generator 33 connected to the wireless transmitter 31, and a snore-stopping function signal generator 34. In this embodiment, the wireless controller 3 is a smart mobile communication device. The high/low adjustment signal generator 32 includes a “rise” icon 321 and a “lower” icon 322 generated by an application software and displayed on a touch screen 35 of the wireless controller 3. Likewise, the sensitivity adjustment signal generator 33 includes a “sensitivity adjustment” icon 330 generated by the application software and displayed on the touch screen 35. Likewise, the snore-stopping function signal generator 34 includes a “stop snore” icon 340 generated by the application software and displayed on the touch screen 35.

In use, an operator firstly executes the application software. The wireless transmitter 31 of the wireless controller 3 searches and connects with the wireless receiver 21 of the wired controller 2 via a blue-tooth wireless transmission interface or a wireless local area network interface. After connection, corresponding icons of the high/low adjustment signal generator 32, the sensitivity adjustment signal generator 33, and the snore-stopping function signal generator 34 are generated on the display 35. Thus, the operator can use the “rise” icon 321 and the “lower” icon 322 by touch control, such that the high/low adjustment signal generator 32 can generate the high/low adjustment control signal which is wirelessly transmitted to the wireless receiver 21 of the wired controller 2. When the wireless receiver 21 receives the high/low adjustment control signal, the control valve 23 and the air pump 24 are activated to inflate or deflate the bladder 12 via the air tube 231 according to the high/low adjustment control signal, thereby adjusting the height of the body 11 of the pillow 1. Furthermore, the operator can touch the “sensitivity adjustment” icon 330 and the “stop snore” icon 340 to send a sensitivity adjustment signal and a snore-stopping function signal to the wireless receiver 21. When the wireless receiver 21 receives the sensitivity adjustment signal, the sensitivity of the sound sensor 25 is adjusted, and the sound sensor 25 is activated. When the wireless receiver 21 receives the snore-stopping function signal, the snore-stopping function controller 27 controls the control valve 23 to proceed with a single cycle of inflation or deflation of the bladder 12, keeping the respiratory tract of the user of the pillow 1 unobstructed and stopping snore.

Furthermore, when the wireless controller 3 executing the application software is connected to the wired controller 2, the touch screen 35 of the wireless controller 3 shows a “disconnect” icon 36 and an “exit” icon 37 to permit disconnection and to permit exit of the application software, respectively.

In view of the foregoing, the remotely operated snore-stopping pillow according to the present invention solves the problems of the prior art and improves the effects. Specifically, the wireless controller 3 wirelessly transmits control signals to the wired controller 2, such that the user can remotely control the height of the pillow 1 and can activate the sound sensor 25. Furthermore, the it is not necessary to place the wired controller 2 near the user of the pillow 1, reducing the affect by electromagnetic waves and avoiding interruption of the sleep of the user. Thus, the pillow 1 can be used in medical therapy. Furthermore, when the user of the pillow 1 snores during sleep, the operator of the wireless controller 3 can adjust the sensitivity of the sound sensor 25 to avoid abnormal activation by the surround sound. Furthermore, it is not necessary to place the wireless controller 3 near the wired controller 2, avoiding disturbance to the user of the pillow 1 and helping the user sleep.

Although specific embodiments have been illustrated and described, numerous modifications and variations are still possible without departing from the scope of the invention. The scope of the invention is limited by the accompanying claims.

1. A remotely operated snore-stopping pillow comprising:
a pillow including a body receiving a bladder;
a wired controller including a wireless receiver, a high/low controller for the pillow, and a control valve, with the wireless receiver connected to the high/low controller, with the control valve connected to an air pump and a sound sensor, with the sound sensor connected to a snore-stopping function controller, with the high/low controller connected to the control valve, with the control valve connected to the bladder of the pillow via an air tube; and
a wireless controller including a wireless transmitter, a high/low adjustment signal generator connected to the wireless transmitter, and a snore-stopping function signal generator.

2. The remotely operated snore-stopping pillow as claimed in claim 1, wherein the wireless receiver of the wired controller and the wireless transmitter of the wireless controller transmit control signals via a blue-tooth wireless transmission interface.

3. The remotely operated snore-stopping pillow as claimed in claim 1, wherein the wireless receiver of the wired controller and the wireless transmitter of the wireless controller transmit control signals via a wireless local area network interface.

4. The remotely operated snore-stopping pillow as claimed in claim 1, wherein the wireless controller is a smart mobile communication device, and the high/low adjustment signal generator and the snore-stopping function signal generator are generated by an application software.

5. The remotely operated snore-stopping pillow as claimed in claim 4, wherein the sound sensor of the wired controller is connected to a sensitivity adjuster, the wireless transmitter is connected to a sensitivity adjustment signal generator, and the sensitivity adjustment signal generator is generated by the application software.
